(19) United States
(12) Patent Application Publication
(10) Pub. No.: US 2013/0020226 A1
(43) Pub. Date: Jan. 24, 2013

(54) Product for parenteral nutrition of obese intensive-care patients

(71) Applicants: Rosa Abele, Konigstein (DE); Ewald Schlotzer, Oberursel (DE)

(72) Inventors: Rosa Abele, Konigstein (DE); Ewald Schlotzer, Oberursel (DE)

(73) Assignee: Fresenius Kabi Deutschland GmbH, Bad Homburg (DE)

(21) Appl. No.: 13/554,356

(22) Filed: Jul. 20, 2012

Related U.S. Application Data
(60) Provisional application No. 61/510,512, filed on Jul. 22, 2011.

(30) Foreign Application Priority Data
Jul. 22, 2011 (EP) 11 175 036.0

(51) Int. Cl. A61K 31/7004; A61K 36/48; B65D 30/22; A61K 38/03; A61P 3/02; A61K 35/60; A61K 36/63

(52) U.S. Cl. 206/568; 514/23; 424/523; 424/757; 424/771; 514/5.5

(57) ABSTRACT

The invention relates to a high-amino-acid, low-lipid and low-carbohydrate parenteral nutrition solution for administration to obese intensive-care patients, and to a three-chamber pouch which comprises the amino acid component, the carbohydrate component and the lipid component of the parenteral nutrition solution separately in three different chambers.

### BACKGROUND OF THE INVENTION

Intensive-care patients are generally not capable of feeding themselves and must therefore be artificially fed. Parenteral nutrition is indicated for many intensive-care patients, since oral or enteral nutrition cannot be effected or cannot be effected to a sufficient extent or is contraindicated. Parenteral nutrition involves administering food via the bloodstream with circumvention of the gastrointestinal tract.

The group of obese intensive-care patients have particular demands with respect to parenteral nutrition. Obesity is present when there is extreme overweight, which is distinguished by an increase in body fat beyond the norm with pathological effects. While obese intensive-care patients generally do not differ from normal-weight or underweight intensive-care patients with regard to the indication for parenteral nutrition, complications occur more frequently in parenterally fed obese intensive-care patients. In addition, the severity of the complications occurring in parenterally fed intensive-care patients is often greater, for example in comparison with normal-weight parenterally fed intensive-care patients.

This can be partly attributed to the fact that adequate parenteral nutrition of obese intensive-care patients using conventional parenteral nutrition solutions of the prior art can only be achieved with great difficulty, since these solutions are essentially tailored to the needs of normal-weight or underweight patients.

However, the needs of obese intensive-care patients with respect to diet composition differ greatly from those of underweight or normal-weight intensive-care patients. For instance, obese intensive-care patients require other ratios and amounts of the macronutrients amino acids, carbohydrates and lipids, one of the reasons for this being that lipid utilization, but also glucose utilization, is often disrupted in this patient group.

In addition, the demand for micronutrients, such as vitamins and trace elements, can also vary from that of underweight or normal-weight intensive-care patients and is often increased.

If the needs of obese intensive-care patients varying from the needs of underweight or normal-weight intensive-care patients are not considered or not sufficiently considered, this can lead to an increased occurrence of metabolic or infectious complications and thus to increased patient morbidity and mortality.

In the prior art, parenteral nutrition solutions are provided which comprise amino acids, lipids and carbohydrates in particular amounts and ratios. Electrolytes can already be contained in the nutrition solutions or be added thereto prior to administration. Trace elements and vitamins are added to the parenteral nutrition solutions prior to administration or administered separately from the parenteral nutrition solution. These parenteral nutrition solutions of the prior art are not orientated to the needs of obese intensive-care patients, but to the nutrient and energy demand of normal-weight or underweight intensive-care patients.

The parenteral nutrition solution dosing recommendations provided in the prior art are also not designed for the nutrition of obese intensive-care patients. For example, dosing intructions for parenteral nutrition solutions often refer to the body weight of the intensive-care patient.

However, body weight, as a reference value, is not well-suited to determining the required quantity of food for obese patients, since body weight is pathologically increased in obese intensive-care patients. Administration of food on the basis of the actual body weight can therefore lead to macronutrients being massively oversupplied. In addition, for obese intensive-care patients, determining body weight by weighing can often only be achieved with great difficulty.

Although adequate nutrition of obese intensive-care patients with individual solutions, in which amino acids, carbohydrates and lipids are fed via separate containers, is conceivable, this requires complex calculations of the required volumes of nutrition solutions by the prescribing physician and also administration from two or more different containers, and this is associated with an increased expenditure of time by care workers, an increased risk of occurrence of administration and dosing errors, and an increased consumption of nutrition solutions and thus also increased costs, since the volumes contained in the individual containers are not harmonized with one another and residual volumes of the individual solutions have to be discarded.

In view of these disadvantages of parenteral nutrition solutions of the prior art, it is an object of the present invention to provide a parenteral nutrition solution for administration to obese intensive-care patients which considers the particular nutritional needs of this patient group and, at the same time, is simple, safe and economical in terms of handling and dosing.

### DISCLOSURE OF THE INVENTION

The invention encompasses a parenteral nutrition solution for use in administration to an obese intensive-care patient, wherein the nutrition solution comprises, per 1000 ml:
(i) 500 ml-834 ml of an amino acid solution comprising 50 g-83 g of amino acids,
(ii) 167 ml-278 ml of a glucose solution comprising 67 g-111 g of glucose, and
(iii) 83 ml-139 ml of a lipid emulsion comprising 17 g-28 g of lipids.

The parenteral nutrition solution can comprise, per 1000 ml:
(i) 667 ml of an amino acid solution comprising 67 g of amino acids,
(ii) 222 ml of a glucose solution comprising 89 g of glucose, and
(iii) 111 ml of a lipid emulsion comprising 22 g of lipids.

In a preferred embodiment, the amino acid solution encompassed by the parenteral nutrition solution comprises, per 1000 ml of the amino acid solution, at least one constituent selected from the group consisting of approximately 5 g of isoleucine, approximately 7.4 g of leucine, approximately 9.31 g of lysine acetate (corresponding to approximately 6.6 g of lysine), approximately 4.3 g of methionine, approximately 5.1 g of phenylalanine, approximately 4.4 g of threonine, approximately 2 g of tryptophan, approximately 6.2 g of valine, approximately 12 g of arginine, approximately 3 g of histidine, approximately g of alanine, approximately 11 g of glycine, approximately 11.2 g of proline, approximately 6.5 g of serine, approximately 0.4 g of tyrosine and approximately 1 g of taurine or a combination thereof.

It is likewise preferred that the lipid emulsion encompassed by the parenteral nutrition solution comprises, per 1000 ml of the lipid emulsion, at least one constituent selected from the group consisting of approximately 60 g of soya oil, approximately 60 g of MCTs, approximately 50 g of olive oil and approximately 30 g of fish oil or a combination thereof.

The parenteral nutrition solution can further comprise electrolytes. In a preferred embodiment, the parenteral nutrition solution comprises, per 1000 ml, electrolytes selected from the group consisting of from approximately 32.8 mmol of sodium to approximately 48 mmol of sodium, from approximately 24 mmol of potassium to approximately 36 mmol of potassium, from approximately 4.1 mmol of magnesium to approximately 6.1 mmol of magnesium, from approximately 2 mmol of calcium to approximately 3 mmol of calcium, from approximately 8.2 mmol of phosphate to approximately 15.6 mmol of phosphate, from approximately 0.032 mmol of zinc to approximately 0.048 mmol of zinc, from approximately 4.1 mmol of sulphate to approximately 6.1 mmol of sulphate, from approximately 28.8 mmol of chloride to approximately 43.2 mmol of chloride, from approximately 84.8 mmol of acetate to approximately 127.2 mmol of acetate or a combination thereof.

A particularly preferred parenteral nutrition solution comprises, per 1000 ml, electrolytes selected from the group consisting of from approximately 36.9 mmol of sodium to approximately 45.1 mmol of sodium, from approximately 27 mmol of potassium to approximately 33 mmol of potassium, from approximately 4.6 mmol of magnesium to approximately 5.6 mmol of magnesium, from approximately 2.3 mmol of calcium to approximately 2.8 mmol of calcium, from approximately 9.2 mmol of phosphate to approximately 14.3 mmol of phosphate, from approximately 0.036 mmol of zinc to approximately 0.044 mmol of zinc, from approximately 4.6 mmol of sulphate to approximately 5.6 mmol of sulphate, from approximately 32.4 mmol of chloride to approximately 39.6 mmol of chloride, from approximately 95.4 mmol of acetate to approximately 116.6 mmol of acetate or a combination thereof.

A very particularly preferred parenteral nutrition solution comprises, per 1000 ml, electrolytes selected from the group consisting of from approximately 36.9 mmol of sodium to approximately 45.1 mmol of sodium, from approximately 27 mmol of potassium to approximately 33 mmol of potassium, from approximately 4.6 mmol of magnesium to approximately 5.6 mmol of magnesium, from approximately 2.3 mmol of calcium to approximately 2.8 mmol of calcium, from approximately 10.2 mmol of phosphate to approximately 13 mmol of phosphate, from approximately 0.036 mmol of zinc to approximately 0.044 mmol of zinc, from approximately 4.6 mmol of sulphate to approximately 5.6 mmol of sulphate, from approximately 32.4 mmol of chloride to approximately 39.6 mmol of chloride, from approximately 95.4 mmol of acetate to approximately 116.6 mmol of acetate or a combination thereof.

Even greater preference is given to a parenteral nutrition solution comprising, per 1000 ml, electrolytes selected from the group consisting of approximately 41 mmol of sodium, approximately 30 mmol of potassium, approximately 5.1 mmol of magnesium, approximately 2.5 mmol of calcium, approximately 13 mmol of phosphate, approximately 0.04 mmol of zinc, approximately 5.1 mmol of sulphate, approximately 36 mmol of chloride, approximately 106 mmol of acetate or a combination thereof.

Most preferred is a parenteral nutrition solution comprising, per 1000 ml of the nutrition solution, 41 mmol of sodium, 30 mmol of potassium, 5.1 mmol of magnesium, 2.5 mmol of calcium, 13 mmol of phosphate, 0.04 mmol of zinc, 5.1 mmol of sulphate, 36 mmol of chloride and 106 mmol of acetate.

The parenteral nutrition solution can comprise, per 1000 of energy, 25% to 350 of energy from amino acids, 40% to 450 of energy from glucose and 22% to 300 of energy from lipids. Preferably, the nutrition solution comprises, per 1000 of energy, 28% to 340 of energy from amino acids, 41% to 440 of energy from glucose and 25% to 270 of energy from lipids. Particularly preferably, the nutrition solution comprises, per 1000 of energy, 320 of energy from amino acids, 420 of energy from glucose and 260 of energy from lipids.

The parenteral nutrition solution has an energy content of from 700 kcal to 975 kcal per 1000 ml. Preferably, the nutrition solution has an energy content of from 800 kcal to 880 kcal per 1000 ml. Most preferred is a nutrition solution having an energy content of from 820 kcal to 860 kcal per 1000 ml.

The parenteral nutrition solution can be contained in a three-chamber pouch comprising a first chamber, a second chamber and a third chamber. Here
(i) the first chamber comprises the amino acid solution,
(ii) the second chamber comprises the glucose solution, and
(iii) the third chamber comprises the lipid emulsion,
   wherein the amino acid solution, the glucose solution and the lipid emulsion are combined and mixed prior to use for administration to an obese intensive-care patient.

If the parenteral nutrition solution contained in the three-chamber pouch comprises electrolytes, these can be contained in the first chamber and/or in the second chamber of the three-chamber pouch.

The invention further encompasses a three-chamber pouch comprising a first chamber, a second chamber and a third chamber wherein
the first chamber comprises 750 ml-2250 ml of an amino acid solution containing 56.3 g-281.3 g of amino acids,
the second chamber comprises 250 ml-750 ml of a glucose solution containing 75 g-375 g of glucose, and
the third chamber comprises 125 ml-375 ml of a lipid emulsion containing 18.8 g-93.8 g of lipids,
   wherein the three-chamber pouch comprises at least 50 g of amino acids per 1000 ml, based on the total volume of the first chamber, the second chamber and the third chamber.

Preferably, the three-chamber pouch comprises at least 60 g of amino acids, particularly preferably at least 63 g of amino acids, even more preferably at least 65 g of amino acids, per 1000 ml, based on the total volume of the first chamber, the second chamber and the third chamber.

In a preferred embodiment, the three-chamber pouch comprises a first chamber, a second chamber and a third chamber wherein the first chamber comprises 1500 ml of an amino acid solution containing 135 g-187.5 g of amino acids, preferably 150 g of amino acids, the second chamber comprises 500 ml of a glucose solution containing 150 g-250 g of glucose, preferably 200 g of glucose, and

the third chamber comprises 250 ml of a lipid emulsion containing 37.5 g-62.5 g of lipids, preferably 50 g of lipids,
or wherein
the first chamber comprises 750 ml of an amino acid solution containing 67.5 g-93.8 g of amino acids, preferably 75 g of amino acids,
the second chamber comprises 250 ml of a glucose solution containing 75 g-125 g of glucose, preferably 100 g of glucose,
and the third chamber comprises 125 ml of a lipid emulsion containing 18.8 g-31.3 g of lipids, preferably 25 g of lipids,
or wherein
the first chamber comprises 2250 ml of an amino acid solution containing 202.5 g-281.3 g of amino acids, preferably 225 g of amino acids,
the second chamber comprises 750 ml of a glucose solution containing 225 g-375 g of glucose, preferably 300 g of glucose,
and the third chamber comprises 375 ml of a lipid emulsion containing 56.3 g-93.8 g of lipids, preferably 75 g of lipids.

The parenteral nutrition solution is suitable for administration to an obese intensive-care patient. In a preferred embodiment, the parenteral nutrition solution is administered to an obese intensive-care patient in a hypercatabolic state and/or hypermetabolic state. In a particularly preferred embodiment, the parenteral nutrition solution is administered to an obese intensive-care patient who is critically ill and/or who has a condition selected from the group consisting of trauma, burn, infection, sepsis and surgical operation or a combination thereof.

The parenteral nutrition solution can be administered to an obese intensive-care patient with class I obesity, class II obesity and class III obesity.

In a further preferred embodiment, the amount of the parenteral nutrition solution to be administered daily is determined by a parameter selected from the group consisting of height of the obese intensive-care patient, sex of the obese intensive-care patient, degree of obesity of the obese intensive-care patient or a combination thereof.

If the patient suffers from class I or class II obesity, the parenteral nutrition solution can be administered to the patient in an amount of from 25 ml to 35 ml per kg of ideal body weight (IBW) per day. Preferably, the parenteral nutrition solution is administered to the patient in an amount of from 28 ml to 32 ml per kg of IBW per day, and particularly preferably in an amount of 30 ml per kg of IBW per day.

If the patient suffers from class III obesity, the parenteral nutrition solution can be administered to the patient in an amount of from 32 ml to 42 ml per kg of IBW per day. Preferably, the parenteral nutrition solution is administered to the patient in an amount of from 35 ml to 39 ml per kg of IBW per day, and particularly preferably in an amount of 37 ml per kg of IBW per day.

If the patient is female and suffers from class I or class II obesity, approximately 2100 ml of the parenteral nutrition solution in the case of a height of 180 cm, approximately 1950 ml in the case of a height of 174 cm, approximately 1800 ml in the case of a height of 168 cm, approximately 1650 ml in the case of a height of 163 cm or approximately 1500 ml in the case of a height of 157 cm can be administered to the patient per day. If the patient is female and suffers from class III obesity, approximately 2590 ml of the parenteral nutrition solution in the case of a height of 180 cm, approximately 2405 ml in the case of a height of 174 cm, approximately 2220 ml in the case of a height of 168 cm, approximately 2035 ml in the case of a height of 163 cm or approximately 1850 ml in the case of a height of 157 cm can be administered to the patient per day.

If the patient is male and suffers from class I or class II obesity, approximately 2550 ml of the parenteral nutrition solution in the case of a height of 187 cm, approximately 2400 ml in the case of a height of 182 cm, approximately 2250 ml in the case of a height of 178 cm, approximately 2100 ml in the case of a height of 173 cm, approximately 1950 ml in the case of a height of 168 cm or approximately 1800 ml in the case of a height of 163 cm can be administered to the patient per day. If the patient is male and suffers from class III obesity, approximately 3145 ml of the parenteral nutrition solution in the case of a height of 187 cm, approximately 2960 ml in the case of a height of 182 cm, approximately 2775 ml in the case of a height of 178 cm, approximately 2590 ml in the case of a height of 173 cm, approximately 2405 ml in the case of a height of 168 cm or approximately 2220 ml in the case of a height of 163 cm can be administered to the patient per day.

Further embodiments of the invention are evident from the following detailed description.

### DEFINITIONS

Parenteral nutrition refers to the administration of food (for example, suitable nutrition solutions) into the bloodstream, i.e. with circumvention of the gastrointestinal tract. Examples thus include intravenous injection or infusion and also intra-arterial injection or infusion. Preferably, the parenteral nutrition solution is administered through a central venous catheter.

Intra-arterial is understood to mean administration into an arterial blood vessel, and intravenous is understood to mean administration into a venous blood vessel. Injection refers to administration using a syringe. Generally, a bolus is administered. However, continuous injection by means of syringe pumps is also possible. Infusion refers to the continuous administration of the composition into a blood vessel, which for example can be effected via a peripheral or central venous catheter.

A parenteral nutrition solution is a nutrition solution suitable for parenteral nutrition. Owing to its constitution, a parenteral nutrition solution can be administered into the bloodstream of a patient.

Obesity is present when there is extreme overweight, which is distinguished by an increase in body fat beyond the norm with pathological effects. According to the definition by the Word Health Organization (WHO (publisher): Obesity: preventing and managing the global epidemic. Report of a WHO Consultation. In: WHO Technical Report Series. 894, 2000), the body mass index (BMI) is used to determine whether obesity is present. Obesity is present above a body mass index (BMI) of 30 kg/m². A distinction is made between three degrees of severity with respect to obesity (class I, class II or class III obesity), and this subdivision is likewise made using the BMI.

Class I obesity is present at a BMI of 30.0-34.99.
Class II obesity is present at a BMI of 35.0-39.99.
Class III obesity is present at a BMI of 0.

The body mass index (BMI) is a measurement for assessing the body weight of a person in relation to his/her height, and is calculated according to the following formula: BMI=m/1²

In this context, m refers to the body weight in kg and 1 refers to the height in m.

The ideal body weight (IBW) in kg for men and women can be calculated according to the following formulae:

For men: IBW=48.0+(height−152)×1.06

For women: IBW=45.4+(height−152)×0.89

When making the calculation, the height in cm must be used.

An intensive-care patient is a male or female patient who has been admitted to hospital and is provided with intensive care. For the purpose of the present application, an “intensive-care patient” can be a patient for whom parenteral nutrition is medically indicated. Parenteral nutrition can be medically indicated when oral or enteral nutrition cannot be effected or cannot be effected to a sufficient extent, or when oral or enteral nutrition is contraindicated.

The expression “hypercatabolic state” describes excessive catabolism.

“Hypermetabolic state” is understood to mean a rise in metabolism in the entire organism.

The expression “critically ill” encompasses intensive-care patients who suffer from systemic inflammatory response syndrome (SIRS), sepsis or multiple organ dysfunction syndrome (MODS) or for whom there is a risk of developing SIRS, MODS or sepsis.

Systemic inflammatory response syndrome (SIRS) is the term for a generalized systemic inflammatory response to a severe event affecting the body (e.g. trauma). With respect to sepsis, SIRS is the umbrella term, since it describes the clinical syndrome of critically ill patients (independently of the cause). Possible non-infectious triggers include pancreatitis, ischaemia, trauma and tissue damage, haemorrhagic shock or burns. If the SIRS is caused by a detected infection, it is referred to as sepsis. Multiple organ failure frequently occurs as a complication and consequence of SIRS and severe sepsis. Multiple organ dysfunction syndrome (MODS) can occur as a result of shock, sepsis or SIRS, and is distinguished by limitations in organ function, organ dysfunctions and/or organ failures. The number of the organs affected and the severity of the organ injuries which occur can vary.

The expression “approximately”, as used here in relation to numerical values, indicates a deviation from the stated numerical value, the magnitude of which is determined by, inter alia, the accuracy of the method for determining the numerical value. In the present invention, “approximately” preferably indicates a deviation of ±5% from the stated numerical value.

The % of energy of a constituent of the nutrition solution refers to the proportion of the total energy content of the nutrition solution due to the constituent, expressed as a percentage. The total energy content of the nutrition solution is 1000. The energy content of a particular food means the amount of energy which the body can obtain upon degradation of a particular food. Energy content is given in calories (kcal) or joules (J).

A dipeptide is understood to mean a compound consisting of two amino acids which are linked to each other via a peptide bond. The dipeptide can be N(2)-L-alanyl-L-glutamine.

Fish oil is understood to mean oil which is obtained from fish and which contains omega-3 fatty acids. The fish oil can be obtained from sea fishes, for example from high sea fishes. For the purpose of the present application, fish oil is highly purified fish oil which is suitable for parenteral administration in humans.

Medium-chain triglycerides (MCTs) are triglycerides which comprise fatty acid radicals of medium length (a length of 6 to 12 carbon atoms). Examples of MCT fatty acids are caproic acid (C6), caprylic acid (C8), capric acid (C10), and lauric acid (C12), or a combination thereof.

Trace elements include chromium (Cr), cobalt (Co), iodine (I), iron (Fe), copper (Cu), manganese (Mn), molybdenum (Mo), selenium (Se) and zinc (Zn).

Dissolved salts dissociated into positively and negatively charged ions can also be referred to as electrolytes. Electrolytes include sodium, potassium, magnesium, calcium, phosphate, zinc, sulphate, chloride and acetate, for example.

Vitamins include vitamin A, vitamin B1, vitamin B2, vitamin B3, vitamin B5, vitamin B6, vitamin B7, vitamin B9, vitamin B12, vitamin C, vitamin D, vitamin E and vitamin K.

### EMBODIMENTS OF THE INVENTION

The headings used below serve only to provide a better overview and are not to be understood as limiting. Where ranges of values are stated below, each of the individual values encompassed by said range is likewise disclosed.

### Composition of the Parenteral Nutrition Solution

Owing to its specific composition, the parenteral nutrition solution is particularly highly suitable for parenteral nutrition of obese intensive-care patients.

The parenteral nutrition solution comprises only constituents which are suitable for parenteral administration in humans. All constituents used are of a purity at which the respective constituent is suitable for parenteral administration. A person skilled in the art is aware of suitable constituents and their required purity for parenteral administration in humans.

The parenteral nutrition solution comprises an amino acid solution, a glucose solution and a lipid emulsion, and can further comprise electrolytes.

If the parenteral nutrition solution comprises electrolytes, these can be contained in the amino acid solution and/or in the glucose solution.

In the parenteral nutrition solution, the amino acid solution, the glucose solution and the lipid emulsion can be present in a combined and mixed form.

Prior to administration to an obese intensive-care patient, trace elements and/or vitamins can be added to the parenteral nutrition solution. Similarly, electrolytes can be added prior to administration if these are not already contained in the amino acid solution and/or the glucose solution or if additional administration of electrolytes in addition to electrolytes already contained in the parenteral nutrition solution is desired.

Alternatively or additionally, electrolytes, trace elements and/or vitamins can also be administered separately from the parenteral nutrition solution.

### (i) Amino Acid Solution

The amino acid demand of obese intensive-care patients is elevated compared to normal-weight intensive-care patients, and is at least 2 g/kg of body weight (based on the ideal weight of the patient) per day for intensive-care patients with class I or II obesity. For intensive-care patients with class III obesity, the amino acid demand has to be estimated to be even higher at at least 2.5 g/kg of body weight (based on the ideal weight of the patient) per day.

A sufficient supply of amino acids is important, for example, for maintaining the lean body mass, more particularly the muscle mass of the patient. Especially in the case of intensive-care patients with injuries, burns or after a surgical operation, a sufficient supply of amino acids is also of great importance for wound healing. It is therefore crucial that amino acids are administered in a sufficient amount with the parenteral nutrition solution to the obese intensive-care patient.

In this context, the inventors have discovered that a parenteral nutrition solution comprising from 0.050 g to 0.083 g of amino acids per ml is highly suitable for administration to obese intensive-care patients, since, firstly, the elevated amino acid demand of the obese intensive-care patients can be met and, secondly, an excessive rise in end products of protein metabolism, however, does not occur.

It was found that, surprisingly, a parenteral nutrition solution comprising from 0.060 g to 0.083 g of amino acids, more particularly from 0.063 g to 0.080 g of amino acids, per ml is particularly highly suitable for parenteral nutrition of obese intensive-care patients. Preference is given to a parenteral nutrition solution comprising from 0.065 g to 0.075 g of amino acids per ml. Particular preference is given to a parenteral nutrition solution comprising from 0.065 g to 0.070 g of amino acids per ml. Most preferred is a parenteral nutrition solution comprising 0.067 g of amino acids per ml.

In a particularly preferred embodiment, the amino acid solution contained in the parenteral nutrition solution comprises, per 1000 ml of the amino acid solution, at least one constituent selected from the group consisting of: approximately 5 g of isoleucine, approximately 7.4 g of leucine, approximately 9.31 g of lysine acetate (corresponding to approximately 6.6 g of lysine), approximately 4.3 g of methionine, approximately 5.1 g of phenylalanine, approximately 4.4 g of threonine, approximately 2 g of tryptophan, approximately 6.2 g of valine, approximately 12 g of arginine, approximately 3 g of histidine, approximately g of alanine, approximately 11 g of glycine, approximately 11.2 g of proline, approximately 6.5 g of serine, approximately 0.4 g of tyrosine and approximately 1 g of taurine or a combination thereof.

Very particular preference is given to an embodiment in which the amino acid solution contained in the parenteral nutrition solution comprises, per 1000 ml of the amino acid solution, 5 g of isoleucin, 7.4 g of leucine, 9.31 g of lysine acetate (corresponding to 6.6 g of lysine), 4.3 g of methionine, 5.1 g of phenylalanine, 4.4 g of threonine, 2 g of tryptophan, 6.2 g of valine, 12 g of arginine, 3 g of histidine, 14 g of alanine, 11 g of glycine, 11.2 g of proline, 6.5 g of serine, 0.4 g of tyrosine and 1 g of taurine.

In a further preferred embodiment, amino acids in the form of dipeptides can be added to the parenteral nutrition solution prior to administration to an obese intensive-care patient, in addition to the amino acids contained in the parenteral nutrition solution. From 0.01 g to 0.04 g of dipeptide, preferably from 0.015 g to 0.030 g of dipeptide, most preferably from 0.02 g to 0.025 g of dipeptide can be added to the parenteral nutrition solution, per ml. In a particularly preferred embodiment, the dipeptide is N(2)-L-alanyl-L-glutamine.

### (ii) Glucose Solution

The parenteral nutrition solution has a comparatively low glucose content which is lower than that of high-amino-acid parenteral nutrition solutions known from the prior art.

In the parenteral nutrition solution for administration to obese intensive-care patients, carbohydrates are provided in the form of glucose. The parenteral nutrition solution comprises from 0.067 g to 0.111 g of glucose per ml. Preferably, the parenteral nutrition solution comprises from 0.070 g to 0.105 g of glucose, or from 0.080 g to 0.095 g of glucose, per ml. Particular preference is given to a parenteral nutrition solution comprising from 0.082 g to 0.093 g of glucose per ml. Most preferred is a parenteral nutrition solution comprising 0.089 g of glucose per ml.

Although the administration of carbohydrates, for example in the form of glucose, is necessary for adequate parenteral nutrition even in obese intensive-care patients, the comparatively low glucose content of the present parenteral tube feeding formula is extremely advantageous in the case of administration to obese intensive-care patients, since this patient group very often already exhibits, or develops during parenteral nutrition, a glucose metabolism disorder in the form of glucose intolerance or manifest diabetes mellitus.

(iii) Lipid Emulsion

Besides differing needs in the supply of amino acids and carbohydrates in parenteral nutrition, obese intensive-care patients also differ from normal-weight or underweight intensive-care patients in lipid demand, since lipid metabolism, like carbohydrate metabolism, is already disrupted in many obese intensive-care patients before the occurrence of the event which makes parenteral nutrition necessary for the patient. In addition, during parenteral nutrition, obese intensive-care patients are exposed to a higher risk than normal-weight or underweight intensive-care patients of developing a lipid metabolism disorder as a result of inadequate parenteral nutrition inconsistent with the particular needs of obese intensive-care patients. Lipid metabolism disorders which are often already present in obese intensive-care patients before the start of parenteral nutrition and which, if they are not already present, often occur as a result of parenteral nutrition include hypertriglyceridaemia. Hypertriglyceridaemia is frequently associated with an increase in transaminases, bilirubin, thrombocytopenia and thrombocytopathy.

The particular composition of the parenteral nutrition solution and the comparatively low energy content provided by lipids can advantageously prevent the occurrence of nutrition-related lipid metabolism disorders in the group of obese intensive-care patients. If a lipid metabolism disorder is already present, intensification of the metabolic disorder can be prevented and derailment of the metabolic disorder due to parenteral nutrition can be avoided.

The parenteral nutrition solution comprises from 0.017 g to 0.028 g of lipids per ml. Preference is given to a parenteral nutrition solution comprising from 0.019 g to 0.026 g of lipids per ml, or comprising from 0.020 g to 0.024 g of lipids. Particularly preferably, the parenteral nutrition solution comprises from 0.021 g to 0.023 g of lipids per ml. Most preferred is a parenteral nutrition solution comprising 0.022 g of lipids per ml; this has been found to be particularly effective.

In the nutrition of obese intensive-care patients, a parenteral nutrition solution in which the comprised lipids have a particular composition has been found to be particularly advantageous. In this preferred embodiment, the lipid emulsion encompassed in the parenteral nutrition solution comprises, per 1000 ml of the lipid emulsion, at least one constituent selected from the group consisting of: approximately 60 g of soya oil, approximately 60 g of MCTs, approximately 50 g of olive oil and approximately 30 g of fish oil or a combination thereof.

Very particular preference is given to a lipid emulsion comprising 60 g of soya oil, 60 g of MCTs, 50 g of olive oil and 30 g of fish oil per 1000 ml.

A lipid emulsion of this composition can prevent the occurrence or deterioration of a pre-existing inflammatory metabolic condition, which is often present in obese intensive-care patients independently of the underlying condition.

### (iv) Energy Content and % of Energy

Parenteral nutrition solutions from the prior art are not orientated to the specific nutritional needs of obese intensive-care patients, one of the reasons being that the solutions have a comparatively high energy content. This is explained, inter alia, by the fact that the parenteral nutrition solutions from the prior art are generally tailored to the pathologically increased energy demand of normal-weight or underweight patients. For this patient group, an energy demand of from 25 kcal to 30 kcal/kg of actual body weight per day is estimated for example.

If obese intensive-care patients are fed accordingly, this results in their being oversupplied with energy which, moreover, originates mainly from carbohydrates and lipids by virtue of the composition of the parenteral nutrition solutions of the prior art. This increases the risk of the occurrence of nutrition-related metabolic and infectious complications in the group of obese intensive-care patients.

With the parenteral nutrition solution for administration to obese intensive-care patients, a nutrition solution is now provided which has an energy content which is particularly suitable for the nutrition of obese intensive-care patients.

The energy content of the parenteral nutrition solution is from 700 kcal to 950 kcal per 1000 ml; preference is given to an energy content of from 750 kcal to 900 kcal per 1000 ml, and also to an energy content of from 800 kcal to 880 kcal per 1000 ml. Particular preference is given to an energy content of from 820 kcal to 860 kcal per 1000 ml; most preferred is an energy content of from 845 kcal to 855 kcal per 1000 ml, for example 850 kcal or 851 kcal per 1000 ml.

Owing to its composition with regard to the ratio of amino acids, glucose and lipids, the parenteral nutrition solution is particularly highly suitable for the nutrition of obese intensive-care patients.

The parenteral nutrition solution can comprise, per 100% of energy, i.e. based on the total energy content of the nutrition solution, 25-35% of energy from amino acids, 40-45% of energy from glucose and 22-30% of energy from lipids. Preference is given to a nutrition solution comprising, per 100% of energy, 28-34% (28%, 29%, 30%, 31%, 32%, 33%, 34%) of energy from amino acids, 41-44% (41%, 42%, 43%, 44%) of energy from glucose and 23-27% (23%, 24%, 25%, 26%, 27%) of energy from lipids. Particular preference is given to an amino acid solution comprising 31-33% (31%, 32%, 33%) of energy from amino acids, 42-44% (42%, 43%, 44%) of energy from glucose and 25-27% (25%, 26%, 27%) of energy from lipids.

Most preferred is a parenteral nutrition solution comprising 32% of energy from amino acids, 42% of energy from glucose and 26% of energy from lipids. Administration of a parenteral nutrition solution of this composition to obese intensive-care patients leads to an unexpectedly low rate of metabolic and infectious complications.

The high energy percentage of amino acids coupled with comparatively low proportions of glucose and lipids makes it possible to effectively prevent overnutrition or malnutrition of obese intensive-care patients and the associated occurrence of nutrition-related metabolic complications such as hyperlipidaemia, hyperglycaemia and glucose intolerance. In the case of obesity-related metabolic disorders already present before the start of parenteral nutrition, intensification of the respective metabolic disorder can be prevented by the particular composition of the parenteral nutrition solution.

By means of the composition of the parenteral nutrition solution, it is also possible to avoid a situation where obese intensive-care patients are undersupplied, especially with amino acids, and to avoid the occurrence of protein energy malnutrition.

Moreover, administration of the parenteral nutrition solution can reduce the occurrence of infection-related complications. Consequently, the morbidity rate and mortality rate of obese intensive-care patients can also be reduced. In addition, shortening of the length of stay in an intensive-care unit can also be achieved.

The composition of the parenteral nutrition solution and its high amino acid content facilitates the mobilization of the endogenous fat of obese intensive-care patients. The parenteral nutrition solution thus permits optimal mobilization of endogenous energy reserves, by means of the high amino acid content and, at the same time, optimal maintenance of endogenous proteins and muscle mass.

### Electrolytes, Trace Elements and Vitamins

The parenteral nutrition solution can contain electrolytes. If the parenteral nutrition solution contains electrolytes, they can be contained in the amino acid solution and/or the glucose solution or be added to the parenteral nutrition solution prior to administration to the obese intensive-care patient. Administration can be carried out in a manner known to a person skilled in the art, for example via an injection port.

In a preferred embodiment, the parenteral nutrition solution comprises, per 1000 ml, electrolytes selected from the group consisting of from approximately 32.8 mmol of sodium to approximately 48 mmol of sodium, from approximately 24 mmol of potassium to approximately 36 mmol of potassium, from approximately 4.1 mmol of magnesium to approximately 6.1 mmol of magnesium, from approximately 2 mmol of calcium to approximately 3 mmol of calcium, from approximately 8.2 mmol of phosphate to approximately 15.6 mmol of phosphate, from approximately 0.032 mmol of zinc to approximately 0.048 mmol of zinc, from approximately 4.1 mmol of sulphate to approximately 6.1 mmol of sulphate, from approximately 28.8 mmol of chloride to approximately 43.2 mmol of chloride, from approximately 84.8 mmol of acetate to approximately 127.2 mmol of acetate or a combination thereof.

A particularly preferred parenteral nutrition solution comprises, per 1000 ml, electrolytes selected from the group consisting of from approximately 36.9 mmol of sodium to approximately 45.1 mmol of sodium, from approximately 27 mmol of potassium to approximately 33 mmol of potassium, from approximately 4.6 mmol of magnesium to approximately 5.6 mmol of magnesium, from approximately 2.3 mmol of calcium to approximately 2.8 mmol of calcium, from approximately 9.2 mmol of phosphate to approximately 14.3 mmol of phosphate, from approximately 0.036 mmol of zinc to approximately 0.044 mmol of zinc, from approximately 4.6 mmol of sulphate to approximately 5.6 mmol of sulphate, from approximately 32.4 mmol of chloride to approximately 39.6 mmol of chloride, from approximately 95.4 mmol of acetate to approximately 116.6 mmol of acetate or a combination thereof.

A very particularly preferred parenteral nutrition solution comprises, per 1000 ml, electrolytes selected from the group consisting of from approximately 36.9 mmol of sodium to approximately 45.1 mmol of sodium, from approximately 27 mmol of potassium to approximately 33 mmol of potassium, from approximately 4.6 mmol of magnesium to approximately 5.6 mmol of magnesium, from approximately 2.3 mmol of calcium to approximately 2.8 mmol of calcium, from approximately 10.2 mmol of phosphate to approximately 13 mmol of phosphate, from approximately 0.036 mmol of zinc to approximately 0.044 mmol of zinc, from approximately 4.6 mmol of sulphate to approximately 5.6 mmol of sulphate, from approximately 32.4 mmol of chloride to approximately 39.6 mmol of chloride, from approximately 95.4 mmol of acetate to approximately 116.6 mmol of acetate or a combination thereof.

Even greater preference is given to a parenteral nutrition solution comprising, per 1000 ml, electrolytes selected from the group consisting of approximately 41 mmol of sodium, approximately 30 mmol of potassium, approximately 5.1 mmol of magnesium, approximately 2.5 mmol of calcium, approximately 13 mmol of phosphate, approximately 0.04 mmol of zinc, approximately 5.1 mmol of sulphate, approximately 36 mmol of chloride, approximately 106 mmol of acetate or a combination thereof.

Most preferred is a parenteral nutrition solution comprising, per 1000 ml of the nutrition solution, 41 mmol of sodium, 30 mmol of potassium, 5.1 mmol of magnesium, 2.5 mmol of calcium, 13 mmol of phosphate, 0.04 mmol of zinc, 5.1 mmol of sulphate, 36 mmol of chloride and 106 mmol of acetate.

Trace elements and/or vitamins can be added to the parenteral nutrition solution prior to administration to the obese intensive-care patient. Administration can be carried out in a manner known to a person skilled in the art, for example via an injection port.

It is preferred that trace elements and vitamins are added in an adequate amount prior to administration, such that any additional administration of trace elements and vitamins to the obese intensive-care patients is no longer necessary.

Preferably, double the amount of the daily amount of trace elements and/or vitamins recommended for healthy normal-weight individuals of the same age and sex can be administered per day with the parenteral nutrition solution. It was found that obese intensive-care patients benefit especially from a parenteral nutrition solution providing such a high amount of trace elements and/or vitamins, since it is precisely obese intensive-care patients who often suffer from a multiple deficiency of trace elements and/or vitamins.

### PREFERRED EMBODIMENTS

The parenteral nutrition solution for administration to an obese intensive-care patient can comprise, per 1000 ml:

(i) 500 ml-834 ml of an amino acid solution comprising 50 g-83 g of amino acids,
(ii) 167 ml-278 ml of a glucose solution comprising 67 g-111 g of glucose, and
(iii) 83 ml-139 ml of a lipid emulsion comprising 17 g-28 g of lipids.

Thus, the parenteral nutrition solution for administration to an obese intensive-care patient comprises, per ml, from 0.05 g to 0.083 g of amino acids, from 0.067 g to 0.111 g of carbohydrates in the form of glucose and from 0.017 g to 0.028 g of lipids.

The parenteral nutrition solution for administration to an obese intensive-care patient can comprise, per 1000 ml:

(i) 500 ml-834 ml of an amino acid solution comprising 60 g-83 g of amino acids,
(ii) 167 ml-278 ml of a glucose solution comprising 67 g-111 g of glucose, and
(iii) 83 ml-139 ml of a lipid emulsion comprising 17 g-28 g of lipids.

Thus, the parenteral nutrition solution for administration to an obese intensive-care patient comprises, per ml, from 0.06 g to 0.083 g of amino acids, from 0.067 g to 0.111 g of carbohydrates in the form of glucose and from 0.017 g to 0.028 g of lipids.

The parenteral nutrition solution can comprise, per ml, from 0.063 g to 0.080 g of amino acids, from 0.070 g to 0.105 g of carbohydrates in the form of glucose and from 0.019 g to 0.026 g of lipids.

Thus, the parenteral nutrition solution for administration to an obese intensive-care patient can, for example, comprise, per 1000 ml:

(i) 500 ml-834 ml of an amino acid solution comprising 63 g-80 g of amino acids,
(ii) 167 ml-278 ml of a glucose solution comprising 70 g-105 g of glucose, and
(iii) 83 ml-139 ml of a lipid emulsion comprising 19 g-26 g of lipids.

Preference is given to a parenteral nutrition solution comprising, per ml, from 0.065 g to 0.075 g of amino acids, from 0.080 g to 0.095 g of carbohydrates in the form of glucose and from 0.020 g to 0.024 g of lipids.

This is the case, for example, in the following preferred parenteral nutrition solution, which comprises, per 1000 ml:

(i) 500 ml-834 ml of an amino acid solution comprising 65 g-75 g of amino acids,
(ii) 167 ml-278 ml of a glucose solution comprising 80 g-95 g of glucose, and
(iii) 83 ml-139 ml of a lipid emulsion comprising 20 g-24 g of lipids.

Even greater preference is given to a parenteral nutrition solution for administration to an obese intensive-care patient, which comprises, per 1000 ml:

(i) 500 ml-834 ml of an amino solution comprising 65 g-70 g of amino acids,
(ii) 167 ml-278 ml of a glucose solution comprising 82 g-93 g of glucose, and
(iii) 83 ml-139 ml of a lipid emulsion comprising 21 g-23 g of lipids.

This particularly preferred embodiment of the parenteral nutrition solution comprises, per ml, from 0.065 g to 0.070 g of amino acids, from 0.082 g to 0.093 g of carbohydrates in the form of glucose and from 0.021 g to 0.023 g of lipids.

Very particular preference is given to a parenteral nutrition solution comprising, per 1000 ml:

(i) 667 ml of an amino acid solution comprising 67 g of amino acids,
(ii) 222 ml of a glucose solution comprising 89 g of glucose, and
(iii) 111 ml of a lipid emulsion comprising 22 g of lipids.

In this very particularly preferred embodiment, the parenteral nutrition solution for administration to an obese intensive-care patient comprises, per ml, 0.067 g of amino acids, 0.089 g of carbohydrates in the form of glucose and 0.022 g of lipids.

In each of the above-mentioned embodiments, the parenteral nutrition solution can comprise electrolytes. These can be contained in the amino acid solution and/or the glucose solution.

### Three-Chamber Pouch

The parenteral nutrition solution can be provided in a three-chamber pouch. The lipid emulsion, the glucose solution and the amino acid solution encompassed in the parenteral nutrition solution can each be contained separately in its own chamber of the three-chamber pouch.

Every three-chamber pouch of the prior art that is suitable for the storage and administration of parenteral nutrition solutions can be used.

The individual chambers (i.e. the first chamber, the second chamber and the third chamber) of the three-chamber pouch can be separated from one another by peel seams. Prior to administration of the parenteral nutrition solution, the peel seams are opened by pressure and the content of the three chambers, the amino acid solution, the glucose solution and the lipid emulsion, are combined and mixed together.

In a first embodiment, the three-chamber pouch comprises a first chamber, a second chamber and a third chamber, with the first chamber comprising 750 ml-2250 ml of an amino acid solution comprising 56.3 g-281.3 g of amino acids, the second chamber comprising 250 ml-750 ml of a glucose solution comprising 75 g-375 g of glucose, and the third chamber comprising 125 ml-375 ml of a lipid emulsion comprising 18.8 g-93.8 g of lipids.

The three-chamber pouch comprises at least 50 g of amino acids, for example from 50 g to 83 g of amino acids, per 1000 ml, based on the total volume of the first chamber, the second chamber and the third chamber. Preference is given to a three-chamber pouch which comprises at least 60 g, for example from 60 g to 83 g, of amino acids per 1000 ml, based on the total volume of the first chamber, the second chamber and the third chamber. Particular preference is given to a three-chamber pouch which comprises at least 63 g, for example from 63 g to 83 g, of amino acids per 1000 ml, based on the total volume of the first chamber, the second chamber and the third chamber. Very particular preference is given to a three-chamber pouch which comprises at least 65 g of amino acids, for example from 65 g to 83 g, per 1000 ml, based on the total volume of the first chamber, the second chamber and the third chamber. Most preferred is a three-chamber pouch which comprises at least 67 g of amino acids, for example from 67 g to 83 g, per 1000 ml, based on the total volume of the first chamber, the second chamber and the third chamber.

In a further, preferred embodiment, the three-chamber pouch comprises a first chamber, a second chamber and a third chamber wherein

the first chamber comprises 1500 ml of an amino acid solution containing 135 g-187.5 g of amino acids, preferably 150 g of amino acids,
the second chamber comprises 500 ml of a glucose solution containing 150 g-250 g of glucose, preferably 200 g of glucose,
and
the third chamber comprises 250 ml of a lipid emulsion containing 37.5 g-62.5 g of lipids, preferably 50 g of lipids.

Also preferred is an embodiment in which the three-chamber pouch comprises a first chamber, a second chamber and a third chamber wherein the first chamber comprises 750 ml of an amino acid solution containing 67.5 g-93.8 g of amino acids, preferably 75 g of amino acids, the second chamber comprises 250 ml of a glucose solution containing 75 g-125 g of glucose, preferably 100 g of glucose,

and the third chamber comprises 125 ml of a lipid emulsion containing 18.8 g-31.3 g of lipids, preferably 25 g of lipids.

Also preferred is an embodiment in which the three-chamber pouch comprises a first chamber, a second chamber and a third chamber wherein

the first chamber comprises 2250 ml of an amino acid solution containing 202.5 g-281.3 g of amino acids, preferably 225 g of amino acids,
the second chamber comprises 750 ml of a glucose solution containing 225 g-375 g of glucose, preferably 300 g of glucose,
and the third chamber comprises 375 ml of a lipid emulsion containing 56.3 g-93.8 g of lipids, preferably 75 g of lipids.

In a particularly preferred embodiment, the three-chamber pouch further comprises electrolytes. Electrolytes can be contained in the first chamber and/or in the second chamber of the three-chamber pouch, i.e. in the amino acid solution and/or in the glucose solution.

Trace elements and/or vitamins can be added to the three-chamber pouch prior to administration of the parenteral nutrition solution to an obese intensive-care patient. Preferably, vitamins and trace elements are added in an adequate amount. If the amino acid solution and the glucose solution do not contain any electrolytes, or if electrolytes are to be administered in addition to electrolytes already contained in the amino acid solution and/or the glucose solution, these can likewise be added to the parenteral nutrition solution prior to administration. Administration is carried out in a manner known to a person skilled in the art, for example via an injection port.

### Advantages

The parenteral nutrition solution composition based on the particular needs of obese intensive-care patients, more particularly the high amino acid content in conjunction with the comparatively low content of glucose and lipids, can, surprisingly and particularly advantageously, lead to a reduction in the frequency of the occurrence of nutrition-related metabolic and infectious complications caused by the parenteral nutrition, and to a reduction in the morbidity and mortality rates of obese intensive-care patients.

The parenteral nutrition solution is low in calories, but high in amino acids. As a result, it is possible to avoid calories being oversupplied and, at the same time, to effectively counter excessive loss of lean body mass, more particularly muscle mass, of the obese intensive-care patient.

The ratio in which amino acids, carbohydrates and lipids are contained in the parenteral nutrition solution has a particularly favourable effect on avoiding or reducing the occurrence of nutrition-related metabolic and infectious complications in this patient group. This is particulary true when the parenteral nutrition solution provides 28% to 340 of energy from amino acids, 41% to 440 of energy from glucose and approximately 25% to 270 of energy from carbohydrates.

The high content of amino acids promotes wound healing and prevents the occurrence of a negative nitrogen balance. At the same time, the parenteral nutrition solution according to the invention can effectively prevent lipids and carbohydrates from being oversupplied to the obese intensive-care patient, as occurs in the case of administration of parenteral nutrition solutions from the prior art, which are consistent with the needs of underweight or normal-weight patients, using normal dosing regimens or instructions.

In the case of administration of the parenteral nutrition solution, glucose metabolism disorders such as hyperglycaemia or glucose intolerance are avoided or attenuated, as are lipid metabolism disorders, such as the occurrence of hypertriglyceridaemia.

The particular composition of the parenteral nutrition solution leads to an interaction, advantageous for the obese intensive-care patient, of the above effects of the high amino acid content with the comparatively low carbohydrate and lipid content of the parenteral nutrition solution which goes beyond a mere additive effect of the three individual effects. This is especially true with regard to the reduction of the nutrition-related complications observed in the obese intensive-care patients, such as hypertriglyceridaemia, hyperglycaemia and disturbance of the amino acid balance, which can be effectively avoided or attenuated by the administration of the parenteral tube feeding formula.

Very particularly effective parenteral nutrition solutions have been found to be those which comprise an amino acid solution, a glucose solution and a lipid emulsion, and which comprise from 0.065 g to 0.070 g of amino acids, from 0.082 g to 0.093 g of carbohydrates in the form of glucose and from 0.021 g to 0.023 g of lipids per ml, wherein the comprised amino acid solution comprises, per 1000 ml of the amino acid solution, approximately 5 g of isoleucine, approximately 7.4 g of leucine, approximately 9.31 g of lysine acetate (corresponding to approximately 6.6 g of lysine), approximately 4.3 g of methionine, approximately 5.1 g of phenylalanine, approximately 4.4 g of threonine, approximately 2 g of tryptophan, approximately 6.2 g of valine, approximately 12 g of arginine, approximately 3 g of histidine, approximately g of alanine, approximately 11 g of glycine, approximately 11.2 g of proline, approximately 6.5 g of serine, approximately 0.4 g of tyrosine and approximately 1 g of taurine, and wherein the comprised lipid emulsion comprises approximately 60 g of soya oil, approximately 60 g of MCTs, approximately 50 g of olive oil and approximately 30 g of fish oil per 1000 ml.

### Administration/Dosing Regimens

The parenteral nutrition solution is outstandingly suitable for parenteral nutrition of obese intensive-care patients.

The parenteral nutrition solution can be administered as the sole food, or in addition to enteral or oral nutrition of the obese intensive-care patient.

In addition, electrolytes, trace elements and/or vitamins can be fed separately from the parenteral nutrition solution to the obese intensive-care patient. However, it is very particularly preferred that electrolytes, trace elements and/or vitamins are contained in the parenteral nutrition solution when administering the parenteral nutrition solution, and so they do not have to be administered separately from the parenteral nutrition solution to the obese intensive-care patient.

The parenteral nutrition solution can be administered as long as sole or supplementary parenteral nutrition is indicated and the intensive-care patient is obese.

For example, the parenteral nutrition solution can be administered over a period of 1 day to several months to an obese intensive-care patient for whom parenteral nutrition is indicated. Owing to its particular composition consistent with the nutritional needs of obese intensive-care patients, the parenteral nutrition solution can be used for longer-term parenteral nutrition of said patients, even if the parenteral nutrition solution is administered as the sole food. The parenteral nutrition solution is thus also suitable for administration over a period of 1 month to 12 months, preferably, too, for administration over a period of 3 months to 12 months, or even 6 months to 12 months.

It is to be expected that the obese patient loses weight as a result of mobilization of endogenous energy reserves while the described parenteral nutrition is administered. The parenteral nutrition solution described here should be replaced by conventional parenteral nutrition once the BMI of the patient falls below 30 kg/m².

Administration can be carried out continuously, i.e. for 24 hours per day without interruption. However, administration can also be carried out discontinuously. In this case, the parenteral nutrition solution is administered to the obese intensive-care patient for, for example, 16 hours per day. For the next 8 hours, no administration is carried out. This is then followed again by administration for 16 hours.

The parenteral nutrition solution comprises an amino acid solution and a glucose solution and a lipid emulsion. These are provided in one pouch, allowing simple dosing. Using the parenteral nutrition solution, it is possible to feed all the required macronutrients (amino acids, carbohydrates and lipids) and also, if desired, all the required electrolytes. Trace elements and vitamins can be added to the parenteral nutrition solution prior to administration.

This form of administration is simple and saves time, and is less prone to miscalculations or errors during actual administration.

In contrast to parenteral nutrition using individual solutions to be administered separately (separate administration of an amino acid solution, a carbohydrate solution and a lipid emulsion), there are no residual volumes of individual nutrition solutions which have to be discarded later. This is also true when the parenteral nutrition solution is provided by means of a three-chamber pouch, since, in this case, although the amino acid solution, glucose solution and lipid emulsion encompassed in the parenteral nutrition solution are stored in separate chambers within the three-chamber pouch prior to administration, the volumes of the amino acid solution, glucose solution and lipid emulsion encompassed in the three-chamber pouch are harmonized with one another such that there are no residual volumes of individual solutions.

If the parenteral nutrition solution is contained in a three-chamber pouch comprising a first chamber, a second chamber and a third chamber, wherein the first chamber comprises the amino acid solution, the second chamber comprises the glucose solution and the third chamber comprises the lipid emulsion, then the amino acid solution, the glucose solution and the lipid emulsion must first be combined and mixed with one another prior to administration of the parenteral nutrition solution to the obese intensive-care patient. Methods for mixing solutions for parenteral nutrition contained in a three-chamber pouch are known to a person skilled in the art. Only after combination and mixing has taken place is the parenteral nutrition solution administered to the obese intensive-care patient.

The obese intensive-care patient can be a patient in a hypercatabolic state and/or hypermetabolic state. The obese intensive-care patient can be a patient who is critically ill and/or be a patient who has a condition selected from the group consisting of trauma, burn, infection, sepsis and surgical operation or a combination thereof.

The obese intensive-care patient can be a patient with class I obesity, class II obesity or class III obesity, and can be male or female.

The amount of the parenteral nutrition solution to be administered daily to the obese intensive-care patient can be determined by a parameter selected from the group consisting of height of the patient, sex of the patient, degree of obesity or a combination thereof.

The degree of obesity can be determined on the basis of the height and the body weight of the patient. Alternatively, the degree of obesity can also be determined on the basis of the amount of body fat and/or the distribution of body fat of the patient by a person skilled in the art.

The administration of the parenteral nutrition solution according to the following regimen has been found to be particularly suitable for parenteral nutrition of obese intensive-care patients:

If the patient suffers from class I or class II obesity, the parenteral nutrition solution can be administered to the patient in an amount of from 25 ml to 35 ml per kg of IBW per day. Preferably, the parenteral nutrition solution is administered to the patient in an amount of from 28 ml to 32 ml per kg of IBW per day, and particularly preferably in an amount of 30 ml per kg of IBW per day.

If the patient suffers from class III obesity, the parenteral nutrition solution can be administered to the patient in an amount of from 32 ml to 42 ml per kg of IBW per day. Preferably, the parenteral nutrition solution is administered to the patient in an amount of from 35 ml to 39 ml per kg of IBW per day, and particularly preferably in an amount of 37 ml per kg of IBW per day.

If it cannot be clearly determined whether a patient suffers from class II or III obesity, and also generally in the transition zone from class II to class III, a dosage between 30 and 37 ml/kg can be selected.

This administration regimen can ensure an adequate supply of nutrients to the patients. At the same time, it is possible to avoid macronutrients being oversupplied.

In a very particularly preferred embodiment, the amount of the parenteral nutrition solution to be administered daily to the obese intensive-care patient can be determined by the height, the sex and the degree of obesity of the obese intensive-care patient.

If the patient is female and suffers from class I or class II obesity, very particular preference is given to administering to the patient, per day, approximately 2100 ml of the parenteral nutrition solution in the case of a height of 180 cm, approximately 1950 ml in the case of a height of 174 cm, approximately 1800 ml in the case of a height of 168 cm, approximately 1650 ml in the case of a height of 163 cm or approximately 1500 ml in the case of a height of 157 cm. If the patient is female and suffers from class III obesity, very particular preference is given to admininstering to the patient, per day, approximately 2590 ml of the parenteral nutrition solution in the case of a height of 180 cm, approximately 2405 ml in the case of a height of 174 cm, approximately 2220 ml in the case of a height of 168 cm, approximately 2035 ml in the case of a height of 163 cm or approximately 1850 ml in the case of a height of 157 cm.

If the patient is male and suffers from class I or class II obesity, very particular preference is given to administering to the patient, per day, approximately 2550 ml of the parenteral nutrition solution in the case of a height of 187 cm, approximately 2400 ml in the case of a height of 182 cm, approximately 2250 ml in the case of a height of 178 cm, approximately 2100 ml in the case of a height of 173 cm, approximately 1950 ml in the case of a height of 168 cm or approximately 1800 ml in the case of a height of 163 cm. If the patient is male and suffers from class III obesity, very particular preference is given to administering to the patient, per day, approximately 3145 ml of the parenteral nutrition solution in the case of a height of 187 cm, approximately 2960 ml in the case of a height of 182 cm, approximately 2775 ml in the case of a height of 178 cm, approximately 2590 ml in the case of a height of 173 cm, approximately 2405 ml in the case of a height of 168 cm or approximately 2220 ml in the case of a height of 163 cm.

Using this very particularly preferred administration regimen, the occurrence of nutrition-related metabolic and infectious complications can be particularly effectively avoided, and the morbidity rate and mortality rate of the obese intensive-care patients can be particularly effectively lowered.

The aforementioned administration regimen concerns administration of the parenteral nutrition solution as the sole parenteral food. If the obese intensive-care patient is additionally fed enterally and/or orally, the amount of energy supplied by the parenteral food as a percentage of the amount of energy supplied by food in total per day is calculated, and only the corresponding percentage is administered according to the aforementioned administration regimen.

### EXAMPLES

### Example 1

### Preparation of a Parenteral Nutrition Solution

### a) The Following Parenteral Nutrition Solutions are Prepared:

Solution

| | A0 | B0 | C0 | D0 | E0 | F0 |
|---|---|---|---|---|---|---|
| Amino acids | 50 | 60 | 83 | 67 | 65 | 63 |
| [g/l] | | | | | | |
| Glucose [g/l] | 67 | 67 | 111 | 89 | 89 | 89 |
| Lipids [g/l] | 17 | 17 | 28 | 22 | 22 | 22 |

### b) The Following Parenteral Nutrition Solutions are Prepared:

Solution

| | A1 | B1 | C1 | D1 | E1 | F1 |
|---|---|---|---|---|---|---|
| Amino acids | 50 | 60 | 83 | 67 | 65 | 63 |
| [g/l] | | | | | | |
| Glucose [g/l] | 67 | 67 | 111 | 89 | 89 | 89 |
| Lipids [g/l] | 17 | 17 | 28 | 22 | 22 | 22 |
| Electrolytes¹ | + | + | + | + | + | + |

¹Solutions A1 to F1 comprise, in contrast to solutions A0 to F0 from example 1a), added electrolytes. In solutions A1 to F1, 41 mmol of sodium, 30 mmol of potassium, 5.1 mmol of magnesium, 2.5 mmol of calcium, 10.2 mmol of phosphate, 0.04 mmol of zinc, 5.1 mmol of sulphate, 36 mmol of chloride and 106 mmol of acetate are added per litre.

### c) Preparation of Solutions A0 to F1 Using a Particularly Preferred Amino Acid Composition

Solutions A0 to F1 are prepared as described under a) and b). In this case, the amino acid source used is an amino acid solution containing, per 1000 ml, 5 g of isoleucine, 7.4 g of leucine, 9.31 g of lysine acetate (corresponding to 6.6 g of lysine), 4.3 g of methionine, 5.1 g of phenylalanine, 4.4 g of threonine, 2 g of tryptophan, 6.2 g of valine, 12 g of arginine, 3 g of histidine, 14 g of alanine, 11 g of glycine, 11.2 g of proline, 6.5 g of serine, 0.4 g of tyrosine and 1 g of taurine.

The solutions thus obtained are given the additional designation “AA” and referred to as A0AA to F1AA.

### d) Preparation of Solutions A0 to F1 Using a Particularly Preferred Lipid Composition

Solutions A0 to F1 are prepared as described under a) and b). In this case, the lipid source used is a lipid emulsion containing, per 1000 ml, 60 g of soya oil, 60 g of MCTs, 50 g of olive oil and 30 g of fish oil.

The solutions thus obtained are given the additional designation “LP” and referred to as A0LP to F1LP.

e) Preparation of Solutions A0 to F1 Using the Particularly Preferred Amino Acid Composition and Lipid composition

Solutions A0 to F1 are prepared as described under a) and b). In this case, the amino acid source used is an amino acid solution containing, per 1000 ml, 5 g of isoleucine, 7.4 g of leucine, 9.31 g of lysine acetate (corresponding to 6.6 g of lysine), 4.3 g of methionine, 5.1 g of phenylalanine, 4.4 g of threonine, 2 g of tryptophan, 6.2 g of valine, 12 g of arginine, 3 g of histidine, 14 g of alanine, 11 g of glycine, 11.2 g of proline, 6.5 g of serine, 0.4 g of tyrosine and 1 g of taurine.

The lipid source used is a lipid emulsion containing, per 1000 ml, 60 g of soya oil, 60 g of MCTs, 50 g of olive oil and 30 g of fish oil.

The solutions thus obtained are given the additional designation “AA-LP” and referred to as A0AA-LP to F1AA-LP.

### Example 2

### Three-Chamber Pouch

The following three-chamber pouches are produced:

| Three-chamber pouch | 1 | 2 | 3 |
|---|---|---|---|
| Total volume of the parenteral | 2250 | 1125 | 3375 |
| nutrition solution [ml] | | | |
| Volume of the solution in | 1500 | 750 | 2250 |
| chamber 1 [ml] | | | |
| Volume of the solution in | 500 | 250 | 750 |
| chamber 2 [ml] | | | |
| Volume of the solution in | 250 | 125 | 375 |
| chamber 3 [ml] | | | |

Chamber 1 of the three-chamber pouch contains an amino acid solution (100 g of amino acids/1), chamber 2 contains a glucose solution (400 g of glucose/1) and chamber 3 contains a lipid emulsion (200 g of lipids/1). There are 67 g/l amino acids, 89 g/l glucose and 22 g/l lipids, based on the total volume of the parenteral nutrition solution in three-chamber pouches 1, 2 and 3. Chamber 1 and/or chamber 2 can additionally contain electrolytes.

The amino acid emulsion can comprise, per 1000 ml, the following composition: 5 g of isoleucine, 7.4 g of leucine, 9.31 g of lysine acetate (corresponding to 6.6 g of lysine), 4.3 g of methionine, 5.1 g of phenylalanine, 4.4 g of threonine, 2 g of tryptophan, 6.2 g of valine, 12 g of arginine, 3 g of histidine, 14 g of alanine, 11 g of glycine, 11.2 g of proline, 6.5 g of serine, 0.4 g of tyrosine and 1 g of taurine.

The lipid emulsion can comprise, per 1000 ml, the following composition: 60 g of soya oil, 60 g of MCTs, 50 g of olive oil and 30 g of fish oil.

### Example 3

### Dosing Regimen

The following table represents an exemplary dosing regimen which can be used to administer the parenteral nutrition solution according to the invention to obese intensive-care patients. The ideal body weight of male and female patients, based on the height of the obese intensive-care patients, can be found in the table; similarly, the recommended dosage of the parenteral nutrition solution based on the height of the patient can be found directly in the table.

Dosing regimen

| | | | Parenteral nutrition solution | |
|---|---|---|---|---|
| Height (cm) | | | according to the invention | |
| Men | Women | IBW* | Class I obesity, | Class III obesity |
| 187 | | 85 | 2550 ml | 3145 ml |
| 182 | | 80 | 2400 | 2960 |
| 178 | | 75 | 2250 | 2775 |
| 173 | 180 | 70 | 2100 | 2590 |
| 168 | 174 | 65 | 1950 | 2405 |
| 163 | 168 | 60 | 1800 | 2220 |
| | 163 | 55 | 1650 | 2035 |
| | 157 | 50 | 1500 | 1850 |

*IBW = ideal body weight

### Example 4

### Examples of Administration

### a) Administration of the Parenteral Nutrition Solution to a 178 Cm Tall Obese Male Intensive-Care Patient.

Using the actual body weight of 150 kg, a BMI of 47.3 can be calcuated for the 178 cm tall patient. The patient therefore has class III obesity. Alternatively, the degree of obesity can also be determined on the basis of the body fat content and/or body fat distribution of the patient, and this does not represent a problem for the hospital staff entrusted with the administration of parenteral nutrition solutions. The table shown in example 3 thus reveals a daily infusion volume of 2775 ml of the parenteral nutrition solution for this patient.

### b) Administration of the Parenteral Nutrition Solution to a 163 cm Tall Obese Female Intensive-Care Patient.

Using the actual body weight of 100 kg, a BMI of 37.6 can be calculated for the 163 cm tall patient. The patient therefore has class II obesity. Alternatively, the degree of obesity can also be determined on the basis of the body fat content and/or body fat distribution of the patient. The table shown in example 3 thus reveals a daily infusion volume of 1800 ml of the parenteral nutrition solution for this patient.

### Example 5

### Effect on Complications, Morbidity and Mortality

Solutions A0-F0 or A1-F1 are administered parenterally according to the dosing regimen shown in example 3 as the sole food to male and female obese intensive-care patients with class I, class II or class III obesity over a period of 2 weeks or 2 months. If one of solutions A0-F0 is administered, electrolytes, trace elements and vitamins are additionally added in an adequate amount to the parenteral nutrition solution prior to administration to the patient. If one of solutions A1-F1 is administered, trace elements and vitamins are addditionally added in an adequate amount to the parenteral nutrition solution prior to administration to the patient.

Durinig the administration period, the occurence of nutrition-related complications such as hyperglycaemia or hypertriglyceridaemia is unlikely. An even nitrogen balance can be reached by the nutrition.

What is observed is a slight reduction in weight of the patient, while mainly preserving the lean body mass.

This leads to a reduced occurrence of metabolic complications. Thus, as a consequence, it is possible to shorten the average length of stay in the intensive-care unit compared to parenterally fed obese intensive-care patients who received a comparative solution consistent with the needs of underweight catabolic patients.

All the solutions are highly suitable for administration to obese intensive-care patients. Solutions having a higher amino acid content (solutions B0-F0 and B1-F1) were particularly effective. Solutions having an amino acid content of 67 g/l, i.e. solutions D0 and D1, perform best.

Particularly good outcomes with respect to a reduction in metabolic complications are achieved when AA-LP type solutions are administered, i.e. A0 AA-LP-F0 AA-LP or A1 AA-LP-F1 AA-LP. What is observed here is a particularly pronounced, more than additive, favourable effect in the form of a reduced frequency in the occurrence of metabolic and infectious complications compared to AA type solutions (A0 AA-F0 AA or A1 AA-F1 AA) or LP type solutions (A0 LP-F0 LP or A1 LP-F1 LP) alone.

### Example 6

### Comparison

Solutions A1-F1 and A1 AA-LP-F1 AA-LP are administered parenterally according to the dosing regimen shown in example 3 as the sole food to male and female obese intensive-care patients with class I, class II or class III obesity over a period of 2 weeks or 2 months. Trace elements and vitamins are additionally added in an adequate amount to the parenteral nutrition solutions prior to administration to the patient.

In the case of obese intensive-care patients who receive one of solutions A1-F1 and A1 AA-LP-F1 AA-LP, the occurrence and/or the severity of metabolic and infectious complications which occur is lower than in the case of patients who receive a comparative solution comprising an identical amino acid content coupled with a high lipid content of 40 g/l. The effect is particularly pronounced when one of solutions A1 AA-LP-F1 AA-LP is administered, especially in the case of solution D1 AA-LP.

1. Parenteral nutrition solution for use in administration to an obese intensive-care patient, the nutrition solution comprising, per 1000 ml:
(i) 500 ml-834 ml of an amino acid solution comprising 50 g-83 g of amino acids,
(ii) 167 ml-278 ml of a glucose solution comprising 67 g-111 g of glucose, and
(iii) 83 ml-139 ml of a lipid emulsion comprising 17 g-28 g of lipids.

2. Parenteral nutrition solution for the use according to claim 1, the nutrition solution comprising, per 1000 ml:
(i) 667 ml of an amino acid solution comprising 67 g of amino acids,
(ii) 222 ml of a glucose solution comprising 89 g of glucose, and
(iii) 111 ml of a lipid emulsion comprising 22 g of lipids.

3. Parenteral nutrition solution for the use according to claim 1, wherein the amino acid solution comprises, per 1000 ml of the amino acid solution, at least one constituent selected from the group consisting of: approximately 5 g of isoleucine, approximately 7.4 g of leucine, approximately 9.31 g of lysine acetate (corresponding to approximately 6.6 g of lysine), approximately 4.3 g of methionine, approximately 5.1 g of phenylalanine, approximately 4.4 g of threonine, approximately 2 g of tryptophan, approximately 6.2 g of valine, approximately 12 g of arginine, approximately 3 g of histidine, approximately 14 g of alanine, approximately 11 g of glycine, approximately 11.2 g of proline, approximately 6.5 g of serine, approximately 0.4 g of tyrosine and approximately 1 g of taurine or a combination thereof,
and/or
wherein the lipid emulsion comprises, per 1000 ml of the lipid emulsion, at least one constituent selected from the group consisting of:
approximately 60 g of soya oil, approximately 60 g of MCTs, approximately 50 g of olive oil and approximately 30 g of fish oil or a combination thereof.

4. Parenteral nutrition solution for the use according to claim 1, wherein the parenteral nutrition solution further comprises at least one constituent selected from the group consisting of:
electrolytes, dipeptides, trace elements, vitamins or a combination thereof.

5. Parenteral nutrition solution for the use according to claim 4, wherein the parenteral nutrition solution comprises, per 1000 ml of the nutrition solution, electrolytes selected from the group consisting of from approximately 32.8 mmol of sodium to approximately 48 mmol of sodium, from approximately 24 mmol of potassium to approximately 36 mmol of potassium, from approximately 4.1 mmol of magnesium to approximately 6.1 mmol of magnesium, from approximately 2 mmol of calcium to approximately 3 mmol of calcium, from approximately 8.2 mmol of phosphate to approximately 15.6 mmol of phosphate, from approximately 0.032 mmol of zinc to approximately 0.048 mmol of zinc, from approximately 4.1 mmol of sulphate to approximately 6.1 mmol of sulphate, from approximately 28.8 mmol of chloride to approximately 43.2 mmol of chloride, from approximately 84.8 mmol of acetate to approximately 127.2 mmol of acetate or a combination thereof,
wherein preferably the parenteral nutrition solution comprises, per 1000 ml of the nutrition solution, electrolytes selected from the group consisting of approximately 41 mmol of sodium, approximately 30 mmol of potassium, approximately 5.1 mmol of magnesium, approximately 2.5 mmol of calcium, approximately 13 mmol of phosphate, approximately 0.04 mmol of zinc, approximately 5.1 mmol of sulphate, approximately 36 mmol of chloride, approximately 106 mmol of acetate or a combination thereof.

6. Parenteral nutrition solution for the use according to claim 1, wherein the nutrition solution comprises, per 100% of energy, 25-35% of energy from amino acids, 40-45% of energy from glucose and 22-30% of energy from lipids,
wherein preferably the nutrition solution comprises, per 100% of energy, 28-34% of energy from amino acids, 41-44% of energy from glucose and 25-27% of energy from lipids,
wherein most preferably the nutrition solution comprises, per 100% of energy, 32% of energy from amino acids, 42% of energy from glucose and 26% of energy from lipids.

7. Parenteral nutrition solution for the use according to claim 1, wherein the nutrition solution has an energy content of 700 kcal-975 kcal per 1000 ml,
wherein preferably the nutrition solution has an energy content of 800 kcal to 880 kcal per 1000 ml, wherein most preferably the nutrition solution has an energy content of 820 kcal-860 kcal per 1000 ml.

8. Parenteral nutrition solution for the use according to claim 1, wherein the parenteral nutrition solution is contained in a three-chamber pouch comprising a first chamber, a second chamber and a third chamber, with
(i) the first chamber comprising the amino acid solution,
(ii) the second chamber comprising the glucose solution, and
(iii) the third chamber comprising the lipid emulsion,
wherein the amino acid solution, the glucose solution and the lipid emulsion are combined and mixed prior to use.

9. Parenteral nutrition solution for the use according to claim 1, wherein the intensive-care patient is a patient in a hypercatabolic state and/or hypermetabolic state,
wherein preferably the intensive-care patient is a patient who is critically ill and/or is a patient who has a condition selected from the group consisting of:
trauma, burn, infection, sepsis and surgical operation or a combination thereof.

10. Parenteral nutrition solution for the use according to claim 1, wherein the obesity is selected from the group consisting of:
class I obesity, class II obesity and class III obesity.

11. Parenteral nutrition solution for the use according to claim 1, wherein the amount of the parenteral nutrition solution to be administered daily is determined by a parameter selected from the group consisting of: height of the patient, sex of the patient, degree of obesity or a combination thereof.

12. Parenteral nutrition solution for the use according to claim 1,
wherein, if the patient suffers from class I or class II obesity, from 25 ml to 35 ml of the parenteral nutrition solution per kg of IBW, preferably from 28 ml to 32 ml per kg of IBW, particularly preferably 30 ml per kg of IBW, are administered to the patient per day,
or
wherein, if the patient suffers from class III obesity, from 32 ml to 42 ml of the parenteral nutrition solution per kg of IBW, preferably from 35 ml to 39 ml per kg of IBW, particularly preferably 37 ml per kg of IBW, are administered to the patient per day.

13. Parenteral nutrition solution for the use according to claim 1,
wherein, if the patient is female and suffers from class I or class II obesity, approximately 2100 ml of the parenteral nutrition solution in the case of a height of 180 cm,
approximately 1950 ml in the case of a height of 174 cm, approximately 1800 ml in the case of a height of 168 cm, approximately 1650 ml in the case of a height of 163 cm or approximately 1500 ml in the case of a height of 157 cm are to be administered to the patient per day,
or
wherein, if the patient is female and suffers from class III obesity, approximately 2590 ml of the parenteral nutrition solution in the case of a height of 180 cm, approximately 2405 ml in the case of a height of 174 cm, approximately 2220 ml in the case of a height of 168 cm, approximately 2035 ml in the case of a height of 163 cm or approximately 1850 ml in the case of a height of 157 cm are to be administered to the patient per day,
or
wherein, if the patient is male and suffers from class I or class II obesity, approximately 2550 ml of the parenteral nutrition solution in the case of a height of 187 cm, approximately 2400 ml in the case of a height of 182 cm, approximately 2250 ml in the case of a height of 178 cm, approximately 2100 ml in the case of a height of 173 cm, approximately 1950 ml in the case of a height of 168 cm or approximately 1800 ml in the case of a height of 163 cm are to be administered to the patient per day,
or
wherein, if the patient is male and suffers from class III obesity, approximately 3145 ml of the parenteral nutrition solution in the case of a height of 187 cm, approximately 2960 ml in the case of a height of 182 cm, approximately 2775 ml in the case of a height of 178 cm, approximately 2590 ml in the case of a height of 173 cm, approximately 2405 ml in the case of a height of 168 cm or approximately 2220 ml in the case of a height of 163 cm are to be administered to the patient per day.

14. Three-chamber pouch comprising a first chamber, a second chamber and a third chamber,
the first chamber comprising 750 ml-2250 ml of an amino acid solution comprising 56.3 g-281.3 g of amino acids,
the second chamber comprising 250 ml-750 ml of a glucose solution comprising 75 g-375 g of glucose,
and
the third chamber comprising 125 ml-375 ml of a lipid emulsion comprising 18.8 g-93.8 g of lipids,
wherein the three-chamber pouch comprises at least 50 g of amino acids per 1000 ml, based on the total volume of the first chamber, the second chamber and the third chamber,
wherein preferably the three-chamber pouch comprises at least 60 g of amino acids, particularly preferably at least 63 g of amino acids, per 1000 ml, based on the total volume of the first chamber, the second chamber and the third chamber.

15. Three-chamber pouch according to claim 14,
wherein the first chamber comprises 1500 ml of an amino acid solution comprising 135 g-187.5 g of amino acids, preferably 150 g of amino acids, the second chamber comprises 500 ml of a glucose solution comprising 150 g-250 g of glucose, preferably 200 g of glucose,
and the third chamber comprises 250 ml of a lipid emulsion comprising 37.5 g-62.5 g of lipids, preferably 50 g of lipids,
or
wherein the first chamber comprises 750 ml of an amino acid solution comprising 67.5 g-93.8 g of amino acids, preferably 75 g of amino acids,
the second chamber comprises 250 ml of a glucose solution comprising 75 g-125 g of glucose, preferably 100 g of glucose,
and the third chamber comprises 125 ml of a lipid emulsion comprising 18.8 g-31.3 g of lipids, preferably 25 g of lipids,
or
wherein the first chamber comprises 2250 ml of an amino acid solution comprising 202.5 g-281.3 g of amino acids, preferably 225 g of amino acids,
the second chamber comprises 750 ml of a glucose solution comprising 225 g-375 g of glucose, preferably 300 g of glucose,
and the third chamber comprises 375 ml of a lipid emulsion comprising 56.3 g-93.8 g of lipids, preferably 75 g of lipids.
